(19) United States
(12) Patent Application Publication
(10) Pub. No.: US 2025/0223279 A1
(43) Pub. Date: Jul. 10, 2025

(54) PROCESS FOR THE PREPARATION OF BELUMOSUDIL MESYLATE AND ITS CRYSTALLINE FORM

(71) Applicant: Glenmark Life Sciences Limited, Solapur (IN)

(72) Inventors: Venkata Raghavendra Acharyulu PALLE, Pune (IN); Pratik PATEL, Vadodara (IN); Gaurav KUMAR, Thane (IN); Subbiah RAMAR, Vadodara (IN); Datta SWARUP, Bankura (IN); Rajendra JAGDHANE, Pune (IN); Navnath GUNJAL, Tal-Sangamner (IN); Kotaiah CHAPALA, Guntur (IN)

(21) Appl. No.: 18/852,702

(22) PCT Filed: Mar. 30, 2023
(86) PCT No.: PCT/IB2023/053174

§ 371 Date: Sep. 30, 2024

(30) Foreign Application Priority Data
Mar. 31, 2022 (IN) 202221019468
Jun. 1, 2022 (IN) 202221031471
Jun. 21, 2022 (IN) 202221035548
Sep. 2, 2022 (IN) 202221050263
Oct. 17, 2022 (IN) 202221059116

(51) Int. Cl. C07D 403/12

(52) U.S. Cl. CPC C07D 403/12

(57) ABSTRACT

The present invention relates to a process for the preparation of belumosudil mesylate and its crystalline form. Further, the present invention relates to a pharmaceutical composition comprising a therapeutically effective amount of the crystalline form of belumosudil mesylate obtained by the process of the present invention.

### PRIORITY

This application claims the benefit of Indian Provisional Applications 202221019468 filed on Mar. 31, 2022, 202221031471 filed on Jun. 1, 2022, 202221035548 filed on Jun. 21, 2022 entitled “Crystalline form of belumosudil mesylate and a process for its preparation”, 202221050263 filed on Sep. 2, 2022 filed on Jun. 1, 2022, entitled “A process for the preparation of crystalline form of belumosudil mesylate and 202221059116 filed on Oct. 17, 2022 entitled “A process for the preparation of belumosudil Mesylate” the contents of which are incorporated herein by reference.

### FIELD OF THE INVENTION

The present invention relates to a process for the preparation of belumosudil mesylate and its crystalline form. Further, the present invention relates to a pharmaceutical composition comprising a therapeutically effective amount of the crystalline form of belumosudil mesylate obtained by the process of the present invention.

### BACKGROUND OF THE INVENTION

Belumosudil, known by its chemical name 2-{3-[4-(1H-indazol-5-ylamino)-2-quinazolinyl]phenoxy}-N-(propan-2-yl)acetamide, is represented by the structure of formula I.

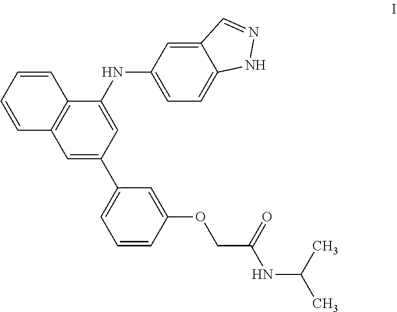 [SMILES: CC(C)NC(=O)COc1cccc(-c2cc(Nc3ccc4[nH]ncc4c3)c3ccccc3c2)c1]

Belumosudil, and its pharmaceutically acceptable salts are described in a published PCT application no. WO2006105081A1 (herein after referred to as WO'081 Application). Belumosudil, sold under the trade name of REZUROCK™ is indicated for the treatment of adult and pediatric patients 12 years and older with chronic graft-versus-host disease (chronic GVHD) after failure of at least two prior lines of systemic therapy. The active pharmaceutical ingredient (API) in REZUROCK™ is belumosudil mesylate, which is known by its chemical name 2-{3-[4-(1H-indazol-5-ylamino)-2-quinazolinyl]phenoxy}-N-(propan-2-yl) acetamide methanesulfonate (1:1).

Various processes for the synthesis of this therapeutically useful compound are known in the art, for instance, the U.S. Pat. No. 9,440,961 (US'961 patent) and Chinese patent No. CN106916145B (CN'145 patent) describe processes for the synthesis of belumosudil or its mesylate salt.

### SUMMARY OF THE INVENTION

The present invention relates to a process for the preparation of belumosudil, a compound of formula I (the “compound I”), or a pharmaceutically acceptable salt thereof,

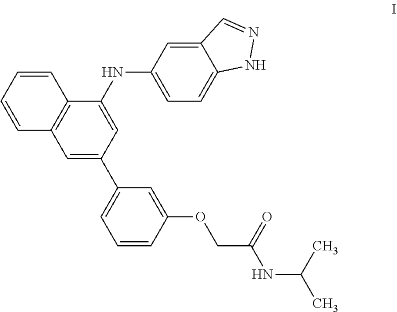 [SMILES: CC(C)NC(=O)COc1cccc(-c2cc(Nc3ccc4[nH]ncc4c3)c3ccccc3c2)c1]

comprising the steps of:
a) reacting chloroacetyl chloride with isopropyl amine to obtain 2-chloro-N-(propan-2-yl)acetamide, a compound of formula II (the “compound II”);

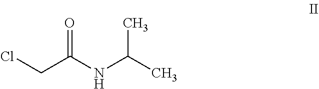

b) reacting the compound II with an alkyl ester of 3-hydroxy benzoate (the “compound III”) in the presence of a base to obtain alkyl ester of 3-[2-(isopropylamino)-2-oxoethoxy]benzoate, a compound of formula IV (the “compound IV”);

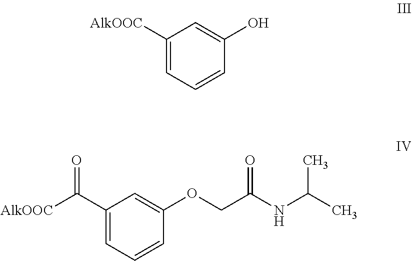

wherein Alk refers to alkyl;
c) reacting the compound IV with an acid or a base to obtain 3-[2-(isopropylamino)-2-oxoethoxy]benzoic acid, the compound of formula V (the “compound V”);

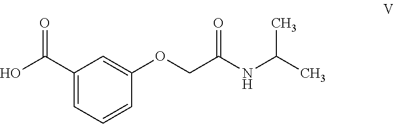 [SMILES: CC(C)NC(=O)COc1cccc(C(=O)O)c1]

d) reacting the compound V with 2-aminobenzamide (the “compound VI”) in the presence of a coupling agent to obtain N-[2-(aminocarbonyl)phenyl]-3-[2-(isopropylamino)-2-oxoethoxy]benzamide, a compound of formula VII (the “compound VII”);

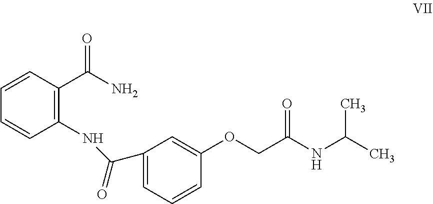 [SMILES: CC(C)NC(=O)COc1cccc(C(=O)Nc2ccccc2C(N)=O)c1]

e) reacting the compound VII with a base to obtain N-isopropyl-2-[3-(4-oxo-3,4-dihydroquinazolin-2-yl)phenoxy]acetamide, a compound of formula VIII (the “compound VIII”);

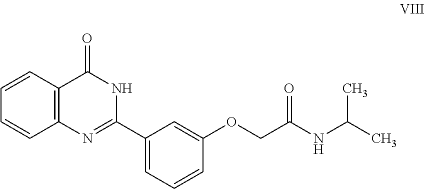 [SMILES: CC(C)NC(=O)COc1cccc(-c2nc3ccccc3c(=O)[nH]2)c1]

f) reacting the compound VIII with a chlorinating agent to obtain 2-[3-(4-chloroquinazolin-2-yl)phenoxy]-N-isopropylacetamide, a compound of formula IX (the “compound IX”);

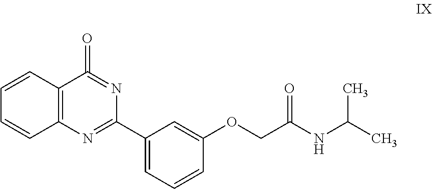

g) reacting the compound IX with 5-aminoindazole to obtain belumosudil, the compound I; and
h) optionally, converting belumosudil to its pharmaceutically acceptable salt.

The present invention also relates to a one-pot process for the preparation of pharmaceutically acceptable salt of belumosudil, a compound of formula I-A (the “compound I-A”),

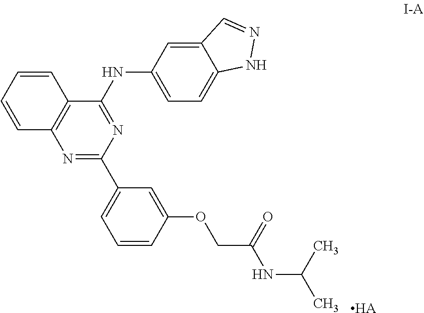

wherein HA is an acid selected from the group consisting of trifluoroacetic acid, methane sulfonic acid, hydrochloric acid, hydrobromic acid, oxalic acid, malonic acid, succinic acid, phosphoric acid, maleic acid, naphthalene-1-sulfonic acid, fumaric acid, ethanedisulfonic acid, mallic acid, sulphuric acid, p-toluene sulphonic acid, besyl acid and the like;
comprising the steps of:
i) reacting the compound IX with 5-aminoindazole to obtain belumosudil, the compound I; and
ii) reacting belumosudil, the compound I, with an acid to obtain the pharmaceutically acceptable salt of belumosudil;
wherein belumosudil, the compound I, obtained in step i) is carried forward without isolation for reaction with an acid in step ii).

The present invention also provides a process for the preparation of crystalline belumosudil mesylate, a compound of formula I-A (the “compound I-A”), wherein HA is methane sulfonic acid,

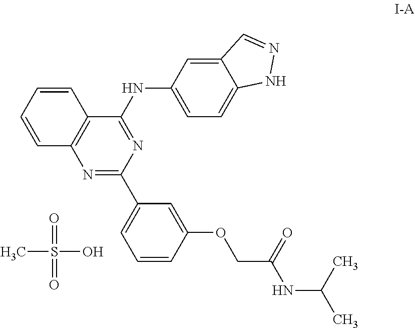

comprising:
(1-a) treating belumosudil mesylate with a mixture of water and a solvent to obtain a reaction mixture;
(1-b) heating the reaction mixture obtained in step (1-a);
(1-c) cooling the reaction mixture obtained in step (1-b); and
(1-d) isolating crystalline belumosudil mesylate from the reaction mixture of step (1-c);
wherein the crystalline belumosudil mesylate is characterised by X-ray powder diffraction (XRPD) spectrum having peak reflections at about 7.1, 17.2, 20.3, 21.5, 22.1, 25.0 and 25.5±0.2° θ.

The present invention provides a process for the preparation of crystalline belumosudil mesylate comprising the steps of:
(L) obtaining a suspension of belumosudil mesylate in a (C₆-C₁₀) hydrocarbon solvent;
(M) heating the suspension obtained in step (L) at a temperature ranging from about 65° C. to reflux temperature of the suspension:
(N) azeotropically distilling the suspension obtained in step (M) at a temperature ranging from about 65° C. to reflux temperature of the suspension;
(O) cooling the suspension of step (N) to a temperature between about 15° C. to about 100° C. to form a precipitate of crystalline belumosudil mesylate; and
(P) isolating the crystalline belumosudil mesylate obtained in step (O);
wherein the process does not comprise the step involving complete dissolution of belumosudil mesylate in the (C₆-C₁₀) hydrocarbon solvent in step (L).

The present invention provides a process for the preparation of crystalline belumosudil mesylate, the “compound I-A, comprising:
(Q) treating belumosudil with methane sulfonic acid in a mixture of water and a solvent to obtain a reaction mixture:
(R) heating the reaction mixture obtained in step (Q) to a suitable temperature:
(S) azeotropically distilling the reaction mixture obtained in step (R);
(T) cooling the reaction mixture obtained in step (S) to obtain crystalline belumosudil mesylate; and
(U) isolating the crystalline belumosudil mesylate from the reaction mixture of step (T):
wherein the crystalline belumosudil mesylate is characterised by X-ray powder diffraction (XRPD) spectrum having peak reflections at about 7.3, 12.9, 13.7, 14.6, 16.6, 17.5, 19.0, 19.6, 20.6 and 26.0±0.2° θ.

### BRIEF DESCRIPTION OF THE DRAWINGS

FIG. 1. Characteristic X-ray Powder Diffraction Pattern (XRPD) of crystalline belumosudil mesylate as obtained in Example 12.

FIG. 2. Characteristic TGA (Thermogravimetric Analysis) thermogram of crystalline belumosudil mesylate as obtained in Example 12.

FIG. 3. Characteristic DSC (Differential Scanning calorimetry) of belumosudil mesylate as obtained in Example 12.

FIG. 4. Characteristic X-ray Powder Diffraction Pattern (XRPD) of crystalline form of belumosudil mesylate as obtained in Example 8.

FIG. 5. Characteristic DSC of crystalline form of belumosudil mesylate as obtained in Example 8.

FIG. 6. Characteristic TGA of crystalline form of belumosudil mesylate as obtained in Example 8.

FIG. 7. ¹³C NMR of crystalline form of belumosudil mesylate as obtained in Example 8.

FIG. 8. Characteristic X-ray Powder Diffraction Pattern (XRPD) of amorphous form of belumosudil as obtained in Example 7.

FIG. 9. Characteristic X-ray Powder Diffraction Pattern (XRPD) of crystalline form of belumosudil mesylate as obtained in Example 9.

FIG. 10. Characteristic TGA (Thermogravimetric Analysis) thermogram of crystalline form of belumosudil mesylate as obtained in Example 9.

FIG. 11. Characteristic X-ray Powder Diffraction Pattern (XRPD) of crystalline belumosudil mesylate as obtained in Example 11.

FIG. 12. Characteristic TGA (Thermogravimetric Analysis) thermogram of crystalline belumosudil mesylate as obtained in Example 11.

FIG. 13. Characteristic DSC (Differential Scanning calorimetry) of belumosudil mesylate as obtained in Example 11.

### DETAILED DESCRIPTION OF THE INVENTION

In one aspect, the present invention provides a process for the preparation of belumosudil, a compound of formula I, or a pharmaceutically acceptable salt thereof,

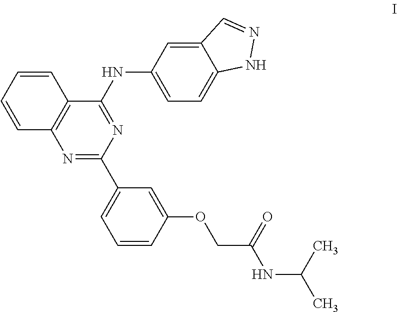 [SMILES: CC(C)NC(=O)COc1cccc(-c2nc(Nc3ccc4[nH]ncc4c3)c3ccccc3n2)c1]

comprising the steps of:
a) reacting chloroacetyl chloride with isopropyl amine to obtain 2-chloro-N-(propan-2-yl)acetamide, a compound of formula II (the “compound II”);

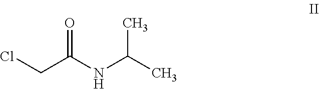

b) reacting the compound II with an alkyl ester of 3-hydroxy benzoate (the “compound III”) in the presence of a base to obtain alkyl ester of 3-[2-(isopropylamino)-2-oxoethoxy]benzoate, a compound of formula IV (the “compound IV”);

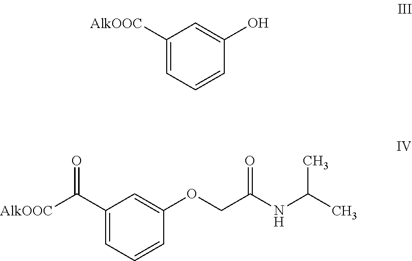

wherein Alk refers to alkyl;
c) reacting the compound IV with an acid or a base to obtain 3-[2-(isopropylamino)-2-oxoethoxy]benzoic acid, the compound of formula V (the “compound V”);

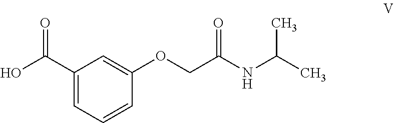 [SMILES: CC(C)NC(=O)COc1cccc(C(=O)O)c1]

d) reacting the compound V with 2-aminobenzamide (the “compound VI”) in the presence of a coupling agent to obtain N-[2-(aminocarbonyl)phenyl]-3-[2-(isopropylamino)-2-oxoethoxy]benzamide, a compound of formula VII (the “compound VII”);

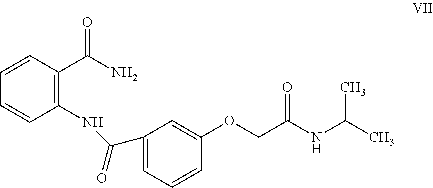 [SMILES: CC(C)NC(=O)COc1cccc(C(=O)Nc2ccccc2C(N)=O)c1]

e) reacting the compound VII with a base to obtain N-isopropyl-2-[3-(4-oxo-3,4-dihydroquinazolin-2-yl)phenoxy]acetamide, a compound of formula VIII (the “compound VIII”);

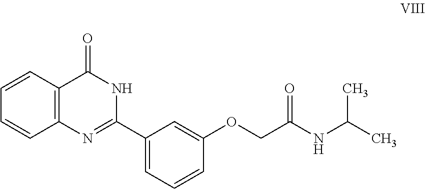 [SMILES: CC(C)NC(=O)COc1cccc(-c2nc3ccccc3c(=O)[nH]2)c1]

f) reacting the compound VIII with a chlorinating agent to obtain 2-[3-(4-chloroquinazolin-2-yl)phenoxy]-N-isopropylacetamide, a compound of formula IX (the “compound IX”);

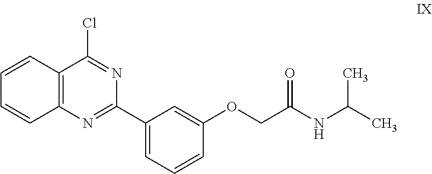 [SMILES: CC(C)NC(=O)COc1cccc(-c2nc(Cl)c3ccccc3n2)c1]

g) reacting the compound IX with 5-amino indazole to obtain belumosudil, the compound I; and
h) optionally, converting belumosudil to its pharmaceutically acceptable salt.

In one embodiment, in step a) of the above process, chloroacetyl chloride is reacted with isopropyl amine in the presence of a solvent to obtain 2-chloro-N-(propan-2-yl)acetamide, the compound II.

In one embodiment, the solvent used in step a) includes, but is not limited to ketone such as acetone, methyl isobutyl ketone, methyl ethyl ketone, acetone, cyclohexanone and the like; hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like: esters such as methyl acetate, ethyl acetate, n-propyl acetate, tert-butyl acetate and the like; nitriles such as acetonitrile, propionitrile, butyronitrile, benzonitrile and the like; amides such as dimethylformamide, dimethyl acetamide and the like; sulfoxides such as dimethyl sulfoxide; water or a mixture thereof.

In one embodiment, the reaction in step a) may be carried out at a temperature ranging from about −10° C. to about 50° C.

As used herein, the term “about” refers to variation through #10 in the defined parameter such as the temperature range or the stirring time used at different steps of processes in the preparation of belumosudil, pharmaceutically acceptable salts of belumosudil including belumosudil mesylate, and the crystalline form of belumosudil mesylate.

In one embodiment, in step b) of the above process, the compound II is reacted with an alkyl ester of 3-hydroxy benzoate, the compound III, in the presence of a base and a solvent to obtain alkyl ester of 3-[2-(isopropylamino)-2-oxoethoxy]benzoate, the compound IV.

In one embodiment, the base used in step b) may be an organic base or an inorganic base.

In one embodiment, the organic base is selected from the group consisting of methylamine, dimethylamine, dipropylamine, tripropylamine, tributylamine, N,N-dimethylcyclohexylamine trimethylamine, ethylamine, propylamine, butylamine, dibutylamine, triethylamine, diisopropylethylamine, cyclopropyl amine, aniline, N,N-dimethyl aniline, N-methylpiperidine, N-methyl morpholine, N-tert-butyl benzyl amine, N-benzylmethylamine and piperidine.

In one embodiment, the inorganic base is selected from the group consisting of hydroxides such as sodium hydroxide, potassium hydroxide and the like; carbonates such as sodium carbonate, potassium carbonate and the like; and bicarbonates such as sodium bicarbonate, potassium bicarbonate and the like.

In one embodiment, the solvent used in step b) includes, but is not limited to ketone such as acetone, methyl isobutyl ketone, methyl ethyl ketone, acetone, cyclohexanone and the like; hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, chloroform, ethylene dichloride, and the like; nitriles such as acetonitrile, and the like; amides such as dimethylformamide, dimethyl acetamide and the like; sulfoxides such as dimethyl sulfoxide; water or mixtures thereof.

In one embodiment, the reaction in step b) may be carried at a temperature ranging from about 15° C. to about reflux temperature of the solvent.

The term “alkyl” as used herein includes a straight or branched chain hydrocarbon containing 1 to 6 carbon atoms. Representative examples of alkyl include, but are not limited to, methyl, ethyl, n-propyl, isopropyl, n-butyl, sec-butyl, tert-butyl, n-pentyl, isopentyl, neopentyl and n-hexyl.

In one embodiment, in step c) of the above process, the compound IV is reacted with an acid or a base to obtain 3-[2-(isopropylamino)-2-oxoethoxy]benzoic acid, the compound V.

The suitable base may be selected from the group consisting of hydroxides such as sodium hydroxide, potassium hydroxide, lithium hydroxide and the like; alkoxides such as sodium methoxide, tert-butoxide and the like.

The suitable acid may be selected from hydrochloric acid, sulphuric acid and the like.

In one embodiment, the reaction in step c) may be carried out in the presence of a suitable solvent.

In one embodiment, the solvent in step c) includes, but is not limited to ketone such as acetone, methyl isobutyl ketone, methyl ethyl ketone, acetone, cyclohexanone and the like; alcoholic solvents such as methanol, ethanol, n-propanol, t-butanol, n-butanol, isopropanol and like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; nitriles such as acetonitrile, propionitrile, butyronitrile, benzonitrile and the like; amides such as dimethylformamide, dimethyl acetamide and the like; sulfoxides such as dimethyl sulfoxide; water or mixtures thereof.

In one embodiment, the reaction in step c) may be carried at a temperature ranging from about 15° C. to about reflux temperature of the solvent.

In one embodiment, step d) of the above process involves reacting the compound V with 2-aminobenzamide in the presence of a coupling agent and optionally in the presence of a solvent and a base, to obtain N-[2-(amino carbonyl)phenyl]-3-[2-(isopropylamino)-2-oxoethoxy]benzamide, the compound VII.

In one embodiment, the base used in step d) may be an organic base selected from the group consisting of dimethylamino pyridine (DMAP), tripropylamine, tributylamine, N,N-dimethylcyclohexylamine trimethylamine, triethylamine, diisopropylethylamine, N,N-dimethyl aniline, N-methylpiperidine, N-methyl morpholine, N-tert-butyl benzyl amine, N-benzylmethylamine and piperidine.

The coupling agent used in step d) is selected from the group consisting of N-Ethyl-N′-(3-dimethylaminopropyl) carbodiimide hydrochloride (EDC·HCl), N,N′-Dicyclohexylcarbodiimide (DCC), 1-Hydroxy benzotriazole (HOBT), 1-[Bis(dimethylamino)methylene]-1H-benzotriazolium 3-Oxide Tetrafluoroborate (TBTU), (2-(1H-benzotriazol-1-yl)-1,1,3,3-tetramethyluronium hexafluoro-phosphate (HBTU), and (1-[Bis(dimethylamino)methylene]-1H-1,2,3-triazolo[4,5-b]pyridinium 3-oxide hexafluorophosphate (HATU).

In one embodiment, the solvent used in step d) includes, but is not limited to, ketone such as acetone, methyl isobutyl ketone, methyl ethyl ketone, acetone, cyclohexanone and the like; alcoholic solvents such as methanol, ethanol, n-propanol, t-butanol, n-butanol, isopropanol and like; hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, propionitrile, butyronitrile, and the like; amides such as dimethylformamide, dimethyl acetamide and the like; sulfoxides such as dimethyl sulfoxide; or mixtures thereof.

In one embodiment, the reaction in step d) may be carried at a temperature ranging from about 15° C. to about reflux temperature of the solvent.

Alternatively, in one embodiment, step d) of the above process, involves activating the carboxy group of the compound V by treating it with an activating reagent to obtain compound of the following formula V-B (the “compound V-B”),

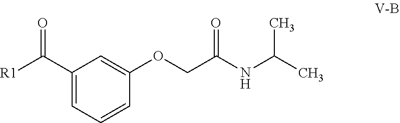

wherein R¹ is an activating group selected from the group consisting of halogen, (C₁₋₆) alkoxy, (C₁₋₆) alkylcarbonyloxy and (C₁₋₆) alkoxycarbonyloxy; wherein halogen is selected from chlorine, bromine or iodine.

In one embodiment, the activating group R¹ may be selected from the group consisting of chloro, methoxy, ethoxy, tert-butoxy, acetoxy and isobutoxycarbonyloxy.

In one embodiment, the reaction in alternative step d) involving activating the carboxy group of the compound V with an activating reagent may be carried out in the presence of a solvent.

In one embodiment, the solvent used in alternative step d) includes, but is not limited to, hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like: nitriles such as acetonitrile, propionitrile, butyronitrile, and the like; amides such as dimethylformamide, dimethyl acetamide and the like; water or mixtures thereof.

In one embodiment, the compound V-B obtained in alternate step d) may be reacted with 2-aminobenzamide (compound VI) to obtain compound VII.

In one embodiment, the reaction in alternative step d) may be carried at a temperature ranging from about −5° C. to about reflux temperature of the solvent.

In one embodiment, in step e) of the above process, the compound VII is reacted with an inorganic base to obtain N-isopropyl-2-[3-(4-oxo-3,4-dihydroquin azolin-2-yl)phenoxy]acetamide, the compound VIII in the presence of a solvent.

In one embodiment, the inorganic base used in step e) is selected from the group consisting of hydroxides such as sodium hydroxide, potassium hydroxide, lithium hydroxide, barium hydroxide and the like; carbonates such as sodium carbonate, potassium carbonate and the like; and bicarbonates such as sodium bicarbonate, potassium bicarbonate and the like.

In one embodiment, the step e) may be carried out in the presence of a solvent.

In one embodiment, the solvent used in step e) includes, but is not limited to, ketone such as acetone, methyl isobutyl ketone, methyl ethyl ketone, acetone, cyclohexanone and the like; alcoholic solvents such as methanol, ethanol, n-propanol, t-butanol, n-butanol, isopropanol and like; hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, propionitrile, butyronitrile, and the like; amides such as dimethylformamide, dimethyl acetamide and the like; sulfoxides such as dimethyl sulfoxide; water or mixtures thereof.

In one embodiment, the reaction in step e) may be carried at a temperature ranging from about 70° C. to about reflux temperature of the solvent.

In one embodiment, in step f) of the above process, the compound VIII is reacted with a chlorinating agent to obtain 2-[3-(4-chloroquinazolin-2-yl)phenoxy]-N-isopropyl acetamide, the compound IX.

In one embodiment, in step f) the chlorinating agent may be selected from the group consisting of thionyl chloride, phosphorous oxychloride and phosphorous pentachloride.

In one embodiment, the reaction in step f) may be carried out in the presence of a solvent.

In one embodiment, the reaction in step f) may be carried out at a temperature of about −20° C. to 20° C.

In one embodiment, the solvent used in step f) includes, but is not limited to, ketone such as acetone, methyl isobutyl ketone, methyl ethyl ketone, acetone, cyclohexanone and the like; alcoholic solvents such as methanol, ethanol, n-propanol, t-butanol, n-butanol, isopropanol and like; hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, propionitrile, butyronitrile, and the like; amides such as dimethylformamide, dimethyl acetamide and the like; sulfoxides such as dimethyl sulfoxide; or mixtures thereof.

In one embodiment, in step g) of the above process, the compound IX is reacted with 5-amino indazole in the presence of a base and a solvent to obtain belumosudil, the compound of formula I.

In one embodiment, the base used in step g) is an organic base, which may be selected from the group consisting of methylamine, dimethylamine, dipropylamine, tripropylamine, tributylamine, N,N-dimethylcyclohexylamine trimethylamine, ethylamine, propylamine, butylamine, dibutylamine, triethylamine, diisopropylethylamine, cyclopropyl amine, aniline, N,N-dimethyl aniline, N-methylpiperidine, N-methyl morpholine, N-tert-butyl benzyl amine, N-benzylmethylamine and piperidine.

In one embodiment the solvent used in step g) includes, but is not limited, to ketone such as acetone, methyl isobutyl ketone, methyl ethyl ketone, acetone, cyclohexanone and the like; alcoholic solvents such as methanol, ethanol, n-propanol, t-butanol, n-butanol, isopropanol and like; hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, propionitrile, butyronitrile and the like; amides such as dimethylformamide, dimethyl acetamide and the like; sulfoxides such as dimethyl sulfoxide; water or mixtures thereof.

In one embodiment, the reaction in step g) may be carried at a temperature ranging from about 70° C. to about reflux temperature of the solvent.

In one embodiment, belumosudil obtained in step g) may be subjected to acid-base purification.

In one embodiment, in step g), the 5-amino indazole, the compound X may be prepared by a process as schematically represented below:

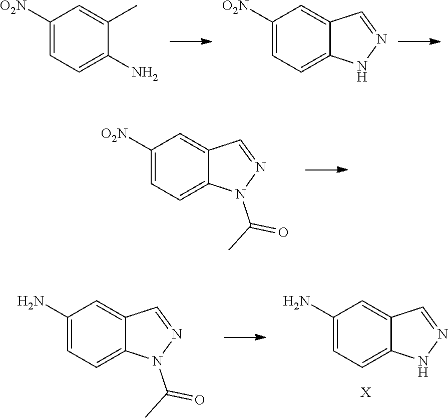

In one embodiment, in step h) of the above process, the compound of formula I is reacted with a suitable acid to obtain the desired pharmaceutically acceptable salt of belumosudil.

In one embodiment, step h) may be carried out in the presence of a solvent. The solvent may be selected from those discussed supra.

In one embodiment, in step h), the acid may be selected from the group consisting of acetic acid, trifluoroacetic acid, hydrochloric acid, hydrobromic acid, nitric acid, oxalic acid, malonic acid, succinic acid, phosphoric acid, maleic acid, naphthalene-1-sulfonic acid, naphthalene-2-sulfonic acid, gallic acid, fumaric acid, ethanedisulfonic acid, citric acid, oxalic acid, malonic acid, mallic acid, sulphuric acid, hydrochloric acid, p-toluene sulphonic acid, methane sulfonic acid, besyl acid and the like.

In one embodiment, the acid is methane sulfonic acid.

In one embodiment, the present invention provides a process for the preparation of belumosudil mesylate, the compound I-A, when HA is MeSO₃H (methanesulfonic acid),

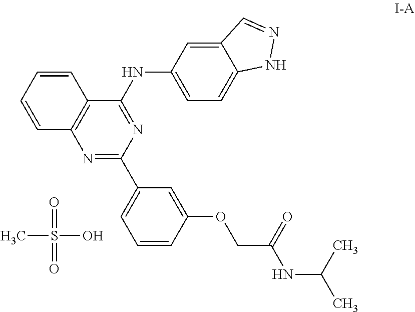

comprising the steps of:
a) reacting chloroacetyl chloride with isopropyl amine to obtain 2-chloro-N-(propan-2-yl)acetamide, the compound II;

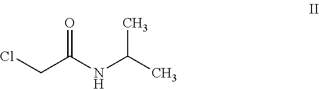

b) reacting the compound II with methyl 3-hydroxy benzoate, the compound of formula III-A (the “compound III-A”) in the presence of a base to obtain methyl 3-[2-(isopropylamino)-2-oxoethoxy]benzoate, a compound of formula IV-A (the “compound IV-A”);

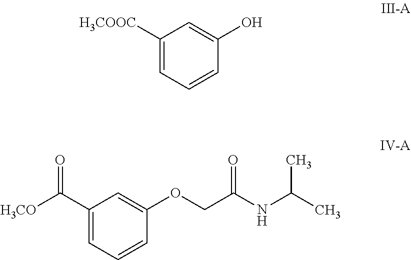

b) reacting the compound IV-A with an acid or a base to obtain 3-[2-(isopropylamino)-2-oxoethoxy]benzoic acid, the compound V;

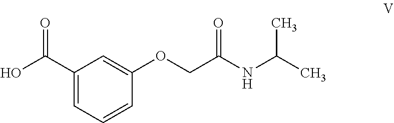 [SMILES: CC(C)NC(=O)COc1cccc(C(=O)O)c1]

d) reacting the compound V with 2-amino benzamide (the compound VI) to obtain N-[2-(aminocarbonyl)phenyl]-3-[2-(isopropylamino)-2-oxoethoxy]benzamide, the compound VII;

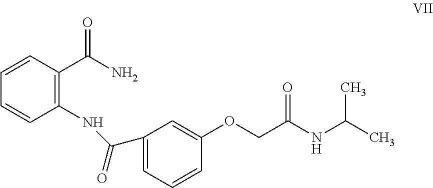 [SMILES: CC(C)NC(=O)COc1cccc(C(=O)Nc2ccccc2C(N)=O)c1]

e) reacting the compound VII with a base to obtain N-isopropyl-2-[3-(4-oxo-3,4-dihydroquinazolin-2-yl)phenoxy]acetamide, the compound VIII;

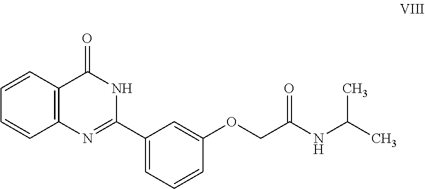 [SMILES: CC(C)NC(=O)COc1cccc(-c2nc3ccccc3c(=O)[nH]2)c1]

f) reacting the compound VIII with a chlorinating agent to obtain 2-[3-(4-haloquinazolin-2-yl)phenoxy]-N-isopropyl acetamide, the compound IX;

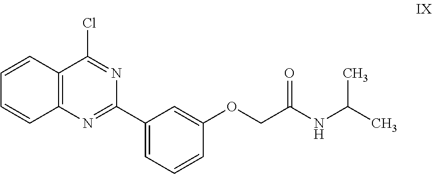 [SMILES: CC(C)NC(=O)COc1cccc(-c2nc(Cl)c3ccccc3n2)c1]

g) reacting the compound IX with 5-amino indazole to obtain belumosudil, the compound I; and
h) reacting belumosudil with methane sulfonic to obtain belumosudil methane sulfonate, the compound I-A.

Belumosudil methane sulfonate salt is also known as belumosudil mesylate. Accordingly, the compound I-A may be interchangeably referred to as belumosudil mesylate or belumosudil methane sulfonate.

In one embodiment, in step h), the reaction of belumosudil with methane sulfonic acid may be carried out in the presence of a solvent.

In one embodiment the solvent used in step h) includes, but is not limited, to ketone such as acetone, methyl isobutyl ketone, methyl ethyl ketone, acetone, cyclohexanone and the like; alcoholic solvents such as methanol, ethanol, n-propanol, t-butanol, n-butanol, isopropanol and like; hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, propionitrile, butyronitrile, and the like; amides such as dimethylformamide, dimethyl acetamide and the like; sulfoxides such as dimethyl sulfoxide; water or mixtures thereof.

In one embodiment, the compound V obtained in the above step d) may be alternatively reacted with thionyl chloride to obtain a compound of formula V-A (the “compound V-A”),

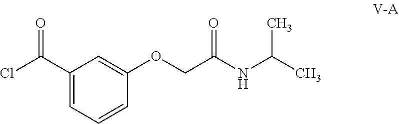 [SMILES: CC(C)NC(=O)COc1cccc(C(=O)Cl)c1]

In one embodiment, the compound V-A thus obtained may be reacted with 2-amino benzamide to obtain N-[2-(amino carbonyl)phenyl]-3-[2-(isopropylamino)-2-oxoethoxy]benzamide, a compound of formula VII (the “compound VII”).

In another aspect, the present invention provides a one-pot process for the preparation of pharmaceutically acceptable salt of belumosudil, a compound of formula I-A (the “compound I-A”);

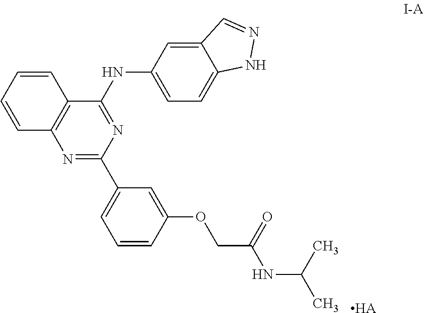

wherein HA is an acid selected from the group consisting of trifluoroacetic acid, methane sulfonic acid, hydrochloric acid, hydrobromic acid, oxalic acid, benzenesulfonic acid, p-toluenesulfonic acid, citric acid, tartaric acid, sulphuric acid, maleic acid, fumaric acid and the like, comprising the steps of:
(i) reacting the compound IX with 5-aminoindazole

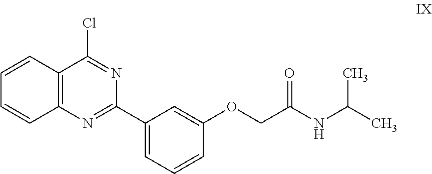 [SMILES: CC(C)NC(=O)COc1cccc(-c2nc(Cl)c3ccccc3n2)c1]

to obtain a reaction mixture containing belumosudil free base, the compound I; and
(ii) reacting the reaction mixture containing the belumosudil free base obtained in step (i) with an acid to obtain the pharmaceutically acceptable salt of belumosudil, the compound I-A:
wherein belumosudil, the compound I, obtained in step i) is carried forward without isolation for reaction with an acid in step ii).

In one embodiment, the present invention provides one-pot process for the preparation of belumosudil mesylate comprising reacting the compound of formula IX with 5-aminoindazole to obtain a reaction mixture comprising belumosudil free base followed by addition of methane sulfonic acid to the resulting reaction mixture to form the belumosudil mesylate.

Thus, the process of the present invention providing one-pot process for the preparation of pharmaceutically acceptable salts of belumosudil such as belumosudil mesylate, is advantageous for large scale synthesis of the pharmaceutically acceptable salts, including the mesylate salt.

WO'081 application discloses belumosudil, a process for its preparation and its intermediates, wherein the process utilizes distillation technique for isolation of crude belumosudil and its purification by preparative HPLC (High Performance Liquid Chromatography). Use of such techniques are not industrially viable.

The present invention avoids use of techniques that involves distillation and purification of crude belumosudil by preparative HPLC thereby providing an industrially viable process, which is cost effective and provides for ease of handling resulting in belumosudil or its pharmaceutically acceptable salt having better purity.

In another aspect, the present invention provides a process for the preparation of crystalline belumosudil mesylate, the compound I-A.

In an embodiment, the present invention provides a process for the preparation of crystalline belumosudil mesylate, the compound I-A, said process comprising the steps of:
(1-a) treating belumosudil mesylate with a mixture of water and a solvent to obtain a reaction mixture:
(1-b) heating the reaction mixture obtained in step (1-a):
(1-c) cooling the reaction mixture obtained in step (1-b); and
(1-d) isolating crystalline belumosudil mesylate from the reaction mixture of step
(1-c); wherein the crystalline belumosudil mesylate is characterised by X-ray powder diffraction (XRPD) spectrum having peak reflections at about 7.1, 17.2, 20.3, 21.5, 22.1, 25.0 and 25.5±0.2° θ.

The belumosudil mesylate used in the step (1-a) of the process for the preparation of its crystalline form, is obtained by a process comprising reacting belumosudil, the compound I, with methanesulfonic acid in the presence of a suitable solvent.

The solvent used in the step (1-a) includes, but is not limited, to ketones such as acetone, methyl isobutyl ketone, methyl ethyl ketone, acetone, cyclohexanone and the like; (C₁-C₆) alcohol such as methanol, ethanol, n-propanol, t-butanol, n-butanol, isopropanol and like; (C₁-C₁₂) hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, propionitrile, butyronitrile and the like; amides such as dimethylformamide, dimethyl acetamide and the like; dimethyl sulfoxide; water or a mixture thereof.

In an embodiment, the solvent used in step (1-a) is (C₁-C₆) alcohol selected from the group consisting of methanol, ethanol, n-propanol, t-butanol, n-butanol, and isopropanol.

The reaction mixture obtained in step (1-a) of the above process is stirred for a period of about 15 minutes to about 3 hours.

In one embodiment, in step (1-b) of the above process, the suitable temperature at which the reaction mixture of step (1-a) is heated ranges from about 40° C. to about reflux temperature of the solvent. The reaction mixture is stirred for a period of 15 minutes to about 3 hours.

In one embodiment, in step (1-c) of the above process, the reaction mixture of step (1-b) is cooled to a temperature ranging from about 0° C. to about 20° C. The reaction mixture is stirred for a period of 15 minutes to about two hours.

In one embodiment, in step (1-d) of the above process, the crystalline belumosudil mesylate from the reaction mixture of step (1-d) is isolated by methods known in the art such as filtration, centrifugation and the like. The isolated crystalline belumosudil mesylate is characterised by X-ray powder diffraction (XRPD) spectrum having peak reflections at about 7.1, 17.2, 20.3, 21.5, 22.1, 25.0 and 25.5±0.2° θ.

In an embodiment, the present invention provides a process for the preparation of crystalline belumosudil mesylate, the compound I-A, comprising the steps of:
(1-aa) treating belumosudil mesylate with a mixture of water and ethanol to obtain a reaction mixture:
(1-bb) heating the reaction mixture obtained in step (1-aa) at a temperature ranging from about 70° C. to about 80° C.:
(1-cc) cooling the reaction mixture obtained in step (1-bb) to a temperature ranging from about 0° C. to about 20° C.; and
(1-dd) isolating crystalline belumosudil mesylate from the reaction mixture of step (1-cc); wherein the crystalline belumosudil mesylate is characterised by X-ray powder diffraction (XRPD) spectrum having peak reflections at about 7.1, 17.2, 20.3, 21.5, 22.1, 25.0 and 25.5±0.2° θ.

In one embodiment, the present invention provides a process for the preparation of crystalline belumosudil mesylate, the compound I-A, characterised by X-ray powder diffraction (XRPD) spectrum having peak reflections at about 7.1, 17.2, 20.3, 21.5, 22.1, 25.0 and 25.5±0.2° θ, wherein the process essentially comprises water.

In one embodiment, the crystalline belumosudil mesylate is characterised by additional peaks at 7.1, 8.3, 15.6, 16.8, 17.2 and 19.5±0.2° θ.

In one embodiment of the present invention, the belumosudil mesylate used in step 1-a) or step 1-aa) of the process for the preparation of crystalline belumosudil mesylate, is obtained by a process comprising the steps of:
A) treating belumosudil with methanesulfonic acid in a solvent to obtain a reaction mixture:
B) heating the reaction mixture of step A):
C) optionally adding an anti-solvent to the reaction mixture of step A or B):
D) cooling the reaction mixture of step A), B) or C):
E) isolating the belumosudil mesylate obtained from step A), B), C) or D).

In one embodiment, in step A) of the above process, the solvent includes, but is not limited, to ketone such as acetone, methyl isobutyl ketone, methyl ethyl ketone, acetone, cyclohexanone; C₁-C₆ alcohol such as methanol, ethanol, n-propanol, t-butanol, n-butanol, isopropanol and the like; C₁-C₁₂ hydrocarbons such as toluene, xylene, chlorobenzene, heptane, hexane, cyclohexane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, propionitrile, butyronitrile and the like; amides such as dimethylformamide, dimethyl acetamide and the like; dimethyl sulfoxide; water or a mixture thereof.

In one embodiment, in step B) of the above process, the reaction mixture is heated to a temperature of about 15° C. to about reflux temperature of the solvent.

In one embodiment, in step C) of the above process, an anti-solvent is added to the reaction mixture of step A) or step B), wherein the anti-solvent includes, but is not limited, to C₁-C₆ hydrocarbons such as xylene, chlorobenzene, heptane, hexane, toluene, cyclohexane and the like; ketones such as acetone, methyl isobutyl ketone, methyl ethyl ketone, acetone, cyclohexanone and the like; ethers such as dimethyl ether, diethyl ether, diisopropyl ether, tert-butyl methyl ether, dibutyl ether, dimethoxyethane, diethoxyethane, tetrahydrofuran, dioxane and the like; water or mixtures thereof.

In one embodiment, in step D) of the above process, the reaction mixture of step A), B) or C) may be cooled to a temperature below 45° C. Preferably, the reaction mixture is cooled to a temperature below 35° C.

In one embodiment, in step E) of the above process, belumosudil mesylate may be isolated by methods known in the art such as filtration or centrifugation.

In one embodiment, in the present invention, the belumosudil mesylate used in step (1-a) or step (1-aa) of the process for the preparation of crystalline belumosudil mesylate, is obtained by a process comprising the steps of:
(F) treating belumosudil with methanesulfonic acid in dimethyl sulfoxide to obtain a reaction mixture:

(G) adding methyl isobutyl ketone to the reaction mixture of step (F); and
(H) isolating belumosudil mesylate from the reaction mixture of step (G).

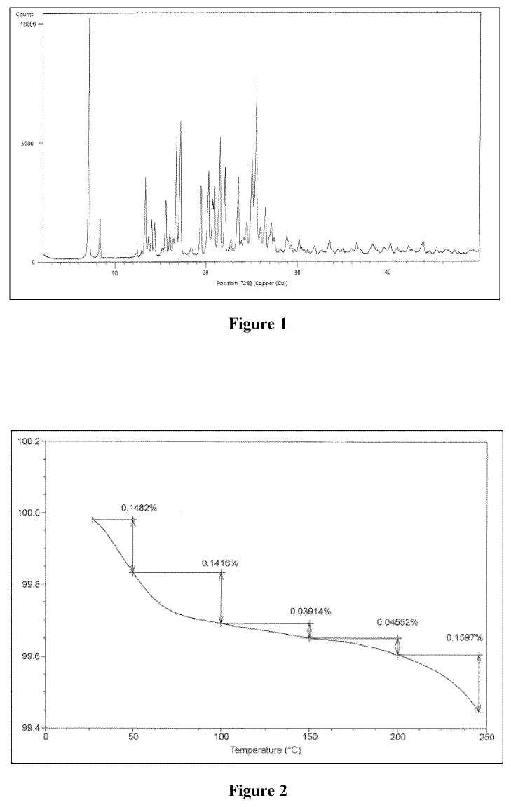

In one embodiment, the crystalline belumosudil mesylate obtained by the process of the present invention, is characterised by X-ray powder diffraction pattern (XRPD) as illustrated in FIG. 1.

In one embodiment, the crystalline belumosudil mesylate obtained by the process of the present invention, is characterised by TGA as illustrated FIG. 2.

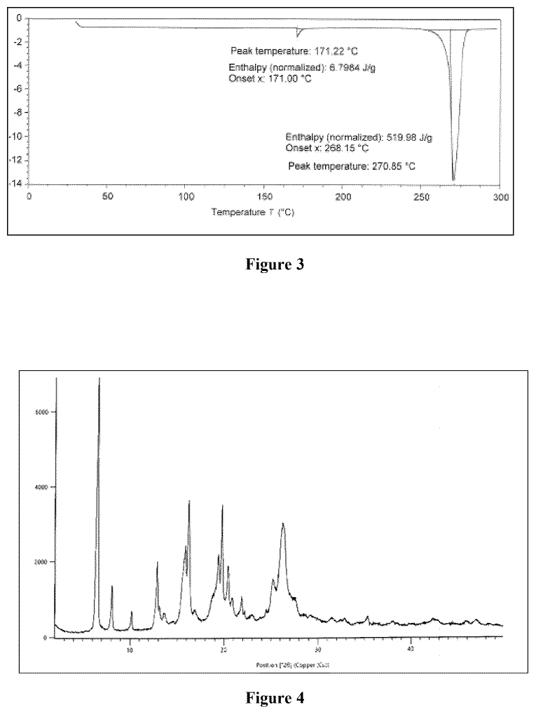

In one embodiment, the crystalline belumosudil mesylate obtained by the process of the present invention, is characterised by DSC as illustrated FIG. 3.

In yet another embodiment, the present invention provides a process for the preparation of crystalline form of belumosudil mesylate comprising the steps of:
(I) dissolving belumosudil mesylate in a solvent to obtain a solution:
(II) obtaining crystalline form of belumosudil mesylate from the solution of step (I); and
(III) isolating the crystalline form of belumosudil mesylate as obtained in step (II).

In an embodiment, the solvent used in step (I) of the process for the preparation of crystalline form of belumosudil mesylate is selected from the group consisting of alcoholic solvents such as methanol, ethanol, n-propanol, t-butanol, n-butanol, isopropanol and like; ketone such as acetone, MIBK, MEK, cyclohexanone and like; ethers such as dimethyl ether, diethyl ether, diisopropyl ether, tert-butyl methyl ether, dibutyl ether, dimethoxyethane, diethoxyethane, tetrahydrofuran, dioxane and the like; esters such as methyl acetate, ethyl acetate, n-propyl acetate, tert-butyl acetate and the like, halogenated hydrocarbons such as dichloromethane, chloroform, ethylene dichloride, and the like, amides such as dimethylformamide, dimethyl acetamide and the like; water and mixtures thereof.

In one embodiment, the step (I) of the process for the preparation of crystalline form of belumosudil mesylate involves dissolving belumosudil mesylate in a solvent selected from the group consisting of alcoholic solvents such as methanol, ethanol, n-propanol, t-butanol, n-butanol, isopropanol and the like or a mixture thereof.

In one embodiment, step (I) may be carried out at a temperature ranging from about 10° C. to about 80° C. Stirring may be continued for any desired time period to achieve a complete dissolution of belumosudil mesylate. The solution may be optionally filtered to get a particle-free solution.

In one embodiment, step (II) of the process for the preparation of crystalline form of belumosudil mesylate, said crystalline form of belumosudil mesylate is obtained from the solution of step (I).

In one embodiment, the step (II) of obtaining crystalline form of belumosudil mesylate may be carried out by:
(i) cooling and stirring the solution obtained in step (I); or
(ii) removing the solvent from the solution obtained in step (I); or
(iii) treating the solution of step (I) with an anti-solvent to form a mixture and optionally, cooling and stirring the resulting mixture.

In an embodiment, the solution obtained in step (I) is cooled to a temperature ranging from about room temperature to about 0° C.

In an embodiment, the solution obtained in step (I) may be stirred over a time period ranging from about 30 minutes to about 10 hours.

In an embodiment, as per step (ii) the removal of solvent from the solution obtained in step (I) may be accomplished by substantially complete evaporation of the solvent; or by concentrating the solution, cooling the solution, if required, and filtering the obtained solid. The solution may also be completely evaporated by using a suitable technique, for example, a rotavapor, a vacuum paddle dryer or in a conventional reactor under vacuum above about 720 mm Hg.

In one embodiment, as per step (iii), the stirring time may range from about 30 minutes to about 10 hours. The resulting mixture may be cooled to a temperature ranging from about room temperature to about 0° C.

In one embodiment, the anti-solvent is selected such that crystalline form of belumosudil mesylate is precipitated out from the solution.

In an embodiment, the anti-solvent is selected from the solvents described herein above.

In one embodiment, in step (III) of the process for the preparation of crystalline form of belumosudil mesylate, said crystalline form of belumosudil mesylate as obtained in step (II) is isolated by any method known in the art. The method, may involve any of techniques, known in the art, including filtration by gravity or by suction, centrifugation, and the like.

In one embodiment, the isolated crystalline form of belumosudil mesylate obtained by a process for the preparation of crystalline belumosudil mesylate, the compound I-A, as per any one of the embodiments described above, may be further dried. Drying may be suitably carried out in an equipment conventionally used in the art for the purpose, such as a tray drier, a vacuum oven, an air oven, a fluidized bed drier, a spin flash drier, a flash drier and the like. The drying may be carried out at a temperature ranging from about room temperature to about 100° C. with or without vacuum. The drying may be carried out for any desired time until the required product quality is achieved. The drying time may vary from about 1 hour to about 25 hours, or longer.

In one embodiment, the present invention provides a process for the preparation of crystalline form of belumosudil mesylate characterised by X-ray powder diffraction pattern (XPRD) having peaks at about 10.1, 16.2 and 18.6±0.2 degrees 2 theta.

In one embodiment, the present invention provides a process for the preparation of crystalline form of belumosudil mesylate characterised by X-ray powder diffraction pattern (XPRD) as illustrated in FIG. 4.

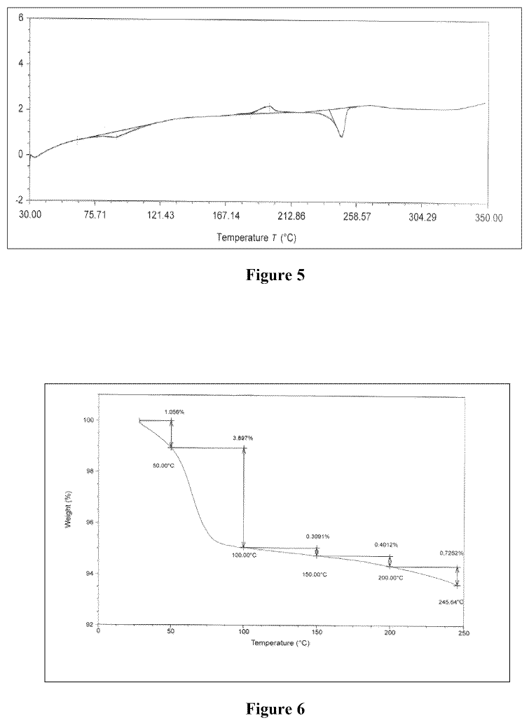

In one embodiment, the present invention provides a process for the preparation of crystalline form of belumosudil mesylate characterised by DSC as illustrated FIG. 5.

In one embodiment, the present invention provides a process for the preparation of crystalline form of belumosudil mesylate characterised by TGA as illustrated FIG. 6.

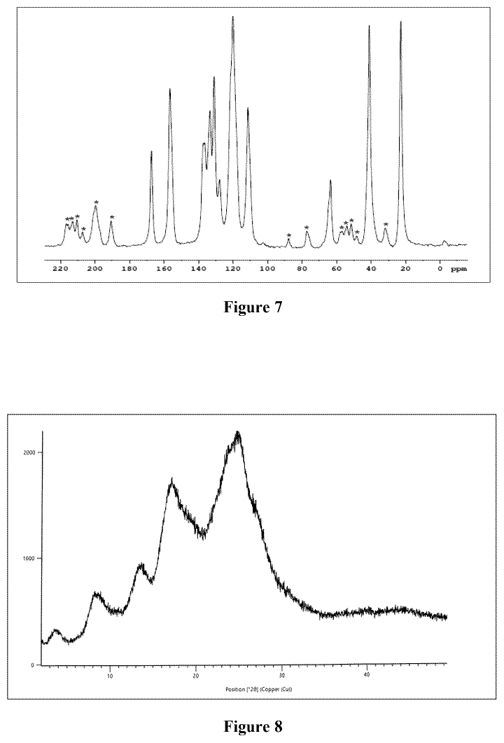

In one embodiment, the present invention provides a process for the preparation of crystalline form of belumosudil mesylate characterised by ¹³CNMR as illustrated FIG. 7.

In one embodiment, provides process for the preparation of crystalline form of belumosudil mesylate which is substantially in accordance with XPRD as illustrated in FIG. 4, substantially in accordance with DSC as illustrated in FIG. 5, substantially in accordance with TGA as illustrated in FIG. 6, substantially in accordance with ¹³C NMR as illustrated in FIG. 7.

In an aspect, the present invention provides a process for preparation of amorphous belumosudil, which may be obtained by reacting compound IX with 5-aminoindazole in the presence of an organic base and a solvent.

In one embodiment, the organic base used in the process for the preparation of amorphous belumosudil is selected from the group consisting of methylamine, dimethylamine, dipropylamine, tripropylamine, tributylamine, N,N-dimethylcyclohexylamine trimethylamine, ethylamine, propylamine, butylamine, dibutylamine, triethylamine, diisopropylethylamine, cyclopropyl amine, aniline, N,N-dimethyl aniline, N-methylpiperidine, N-methyl morpholine, N-tert-butyl benzyl amine, N-benzylmethylamine and piperidine.

In one embodiment, the solvent used in the process for the preparation of amorphous belumosudil includes, but is not limited to ketone such as acetone, methyl isobutyl ketone, methyl ethyl ketone, acetone, cyclohexanone and the like; hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, propionitrile, butyronitrile, benzonitrile and the like; amides such as dimethylformamide, dimethyl acetamide and the like; sulfoxides such as dimethyl sulfoxide; water or mixtures thereof.

In one embodiment, the present invention provides amorphous form of belumosudil as illustrated in FIG. 8.

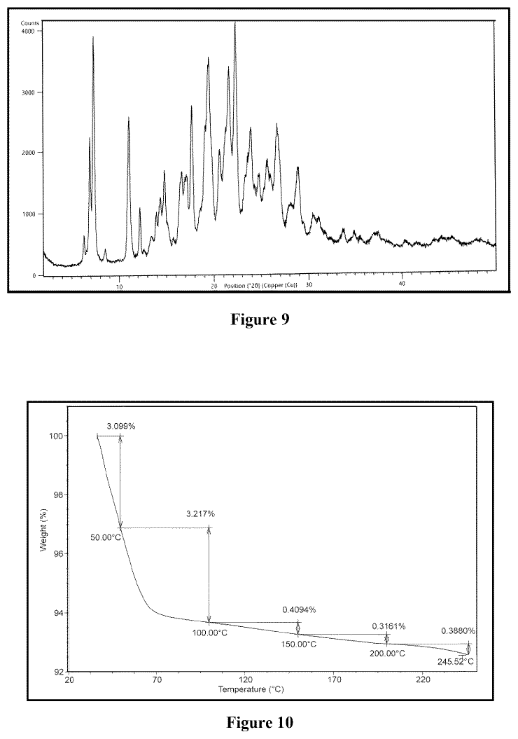

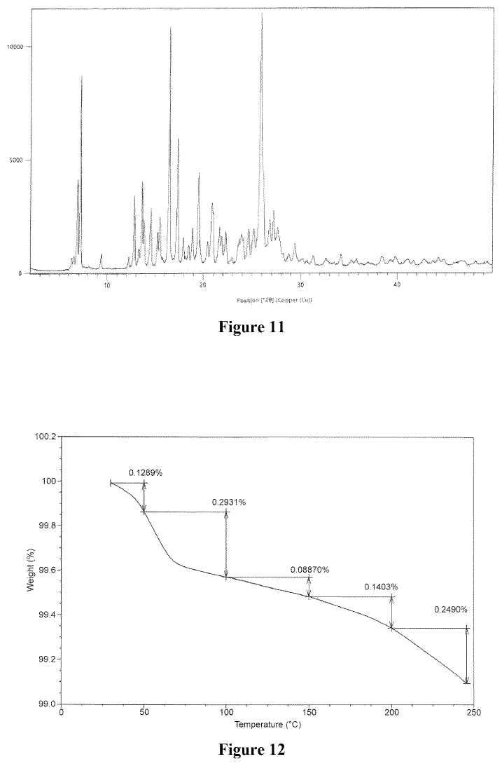

In one embodiment, the present invention provides a process for the preparation of crystalline belumosudil mesylate isolated from 1,4-dioxane, which is substantially in accordance with XPRD as illustrated in FIG. 9 and substantially in accordance with TGA as illustrated in FIG. 10.

In one embodiment, the present invention provides a process for the preparation of crystalline belumosudil mesylate which is isolated from a reaction mixture comprising C₁-C₆ hydrocarbons.

In another embodiment, the present invention provides a process for the preparation of crystalline belumosudil mesylate by treating belumosudil with methanesulfonic acid in a mixture of water and solvent wherein the process comprises the step of azeotropic distillation.

In an embodiment, the present invention provides a process for the preparation of crystalline belumosudil mesylate comprising the steps of:
(L) obtaining a suspension of belumosudil mesylate in a (C₆-C₁₀) hydrocarbon solvent:
(M) heating the suspension obtained in step (L) at a temperature ranging from about 65° C. to reflux temperature of the suspension;
(N) azeotropically distilling the suspension obtained in step (M) at a temperature ranging from about 65° C. to reflux temperature of the suspension:
(O) cooling the suspension of step (N) to a temperature between about 15° C. to about 100° C. to form a precipitate of crystalline belumosudil mesylate; and
(P) isolating the crystalline belumosudil mesylate obtained in step (O).

In an embodiment, the above process does not involve complete dissolution of belumosudil mesylate in the (C₆-C₁₀) hydrocarbon solvent in step (L).

In one embodiment, in the above step (L), the (C₆-C₁₀) hydrocarbon solvent may be selected from the group consisting of hexane, cyclohexane, toluene, o-xylene, p-xylene, m-xylene or a mixture of ortho, meta- and para xylene; and the like.

In one embodiment, in the above step (M), the suspension is heated to a temperature from about 65° C. to about 200° C. temperature of the suspension.

In one embodiment, in the above step (N) water is removed azeotropically at a temperature from about 65° C. to about 200° C.

As used herein, the term, “reflux” refers to a temperature in the range of about 65° C. to about 200° C. or in the range of about 75° C. to about 160° C.

In one embodiment, in the above step (P) the crystalline belumosudil mesylate may be isolated by methods known in the art such as filtration or centrifugation.

In one embodiment, the present invention provides crystalline belumosudil mesylate characterised by X-ray powder diffraction pattern (XRPD) as illustrated in FIG. 11.

In one embodiment, the present invention provides crystalline belumosudil mesylate characterised by TGA as illustrated FIG. 12.

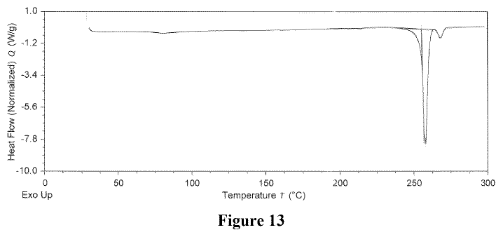

In one embodiment, the present invention provides crystalline belumosudil mesylate characterised by DSC as illustrated FIG. 13.

In one embodiment, the present invention provides a process for the preparation of crystalline belumosudil mesylate, a compound of formula I-A, comprising the steps of:
(Q) treating belumosudil with methane sulfonic acid in a mixture of water and a solvent to obtain a reaction mixture;
(R) heating the reaction mixture obtained in step (Q);
(S) azeotropically distilling the reaction mixture obtained in step (R);
(T) cooling the reaction mixture to obtain crystalline belumosudil mesylate; and
(U) isolating the crystalline belumosudil mesylate from the reaction mixture of step (T);
wherein the crystalline belumosudil mesylate is characterised by X-ray powder diffraction (XRPD) spectrum having peak reflections at about 7.3, 12.9, 13.7, 14.6, 16.6, 17.5, 19.0, 19.6, 20.6 and 26.0±0.2° θ.

In one embodiment, in step (Q) of the above process, either methane sulfonic acid is added to a mixture of belumosudil, water and a solvent, or a mixture of belumosudil, water and a solvent is added to methane sulfonic acid.

In a specific embodiment of the invention, in step (R), methanesulfonic acid is added to a mixture of belumosudil, water and a solvent to obtain a reaction mixture.

The solvent may be selected from the group consisting (C₁-C₆) alcohols such as methanol, ethanol, propanol and the like; (C₁-C₁₂) hydrocarbons such as hexane, heptane, toluene, o-xylene, m-xylene, p-xylene, cyclohexane and the like; amides such as dimethyl formamide, acetamide and the like; dimethyl sulfoxide; or mixtures thereof.

In one embodiment, in step (R) of the above process, the reaction mixture is heated to a temperature of about 45° C. to about reflux temperature of the reaction mixture. Preferably, the reaction mixture is heated to its reflux temperature. The reaction mixture is stirred at the reflux temperature for a period of about 15 minutes to about 3 hr.

In one embodiment, in step (S) of the above process, the reaction mixture of step (S) is cooled to a temperature ranging from about 45° C. to about 65° C.

In one embodiment, in step (U) of the above process, the crystalline belumosudil mesylate is isolated from the reaction mixture of step (T) by methods such as filtration or centrifugation. The crystalline belumosudil mesylate thus isolated from the reaction mixture of step (T) is characterised by X-ray powder diffraction (XRPD) spectrum having peak reflections at about 7.3, 12.9, 13.7, 14.6, 16.6, 17.5, 19.0, 19.6, 20.6 and 26.0±0.2° θ.

In one embodiment, the present invention provides crystalline belumosudil mesylate which is isolated from a reaction mixture comprising (C₁-C₆) hydrocarbons wherein the process comprises the step of azeotropic distillation, and the resulting crystalline belumosudil mesylate is substantially in accordance with the XRPD as illustrated in FIG. 11, or is substantially in accordance with TGA as illustrated in FIG. 12; is substantially in accordance with DSC as illustrated in FIG. 13.

In one embodiment, the present invention provides crystalline belumosudil mesylate which is obtained by treating belumosudil with methanesulfonic acid in a mixture of water and a solvent, wherein the process comprises the step of azeotropic distillation, and the resulting crystalline belumosudil mesylate is substantially in accordance with the XRPD as illustrated in FIG. 11, or is substantially in accordance with TGA as illustrated in FIG. 12; is substantially in accordance with DSC as illustrated in FIG. 13.

It is well known in the pharmaceutical art that impurities that may be generated during the production and storage of a drug substance such as belumosudil mesylate may not only affect the purity, but also affects the efficacy of the drug substance when incorporated in a finished dosage form, and may lead to severe side effects of the drug.

In one embodiment, the processes of the present invention for the preparation of belumosudil mesylate, the compound I-A, provides the compound I-A substantially free of impurities represented by the following compounds of formulae XI to XXII (hereinafter referred to as compounds XI to XXII):

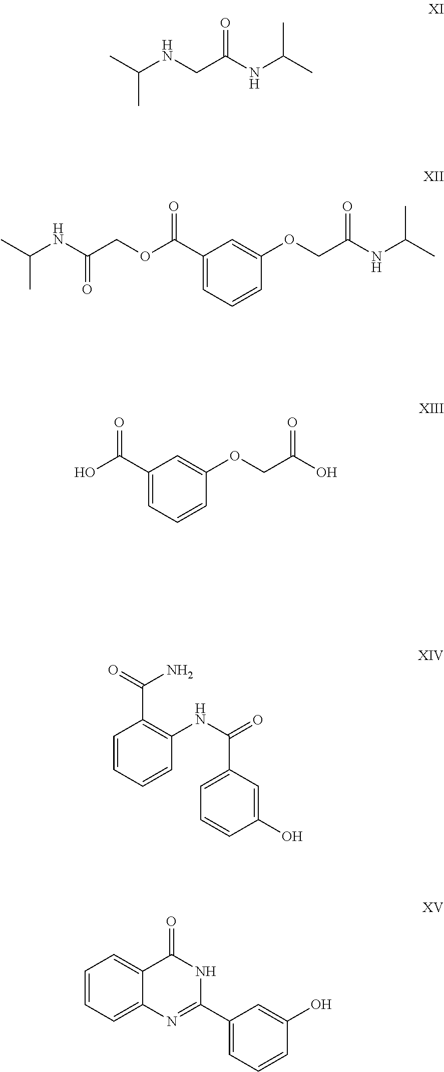 [SMILES: CC(C)NCC(=O)NC(C)C ; CC(C)NC(=O)COC(=O)c1cccc(OCC(=O)NC(C)C)c1 ; O=C(O)COc1cccc(C(=O)O)c1 ; NC(=O)c1ccccc1NC(=O)c1cccc(O)c1 ; O=c1[nH]c(-c2cccc(O)c2)nc2ccccc12]

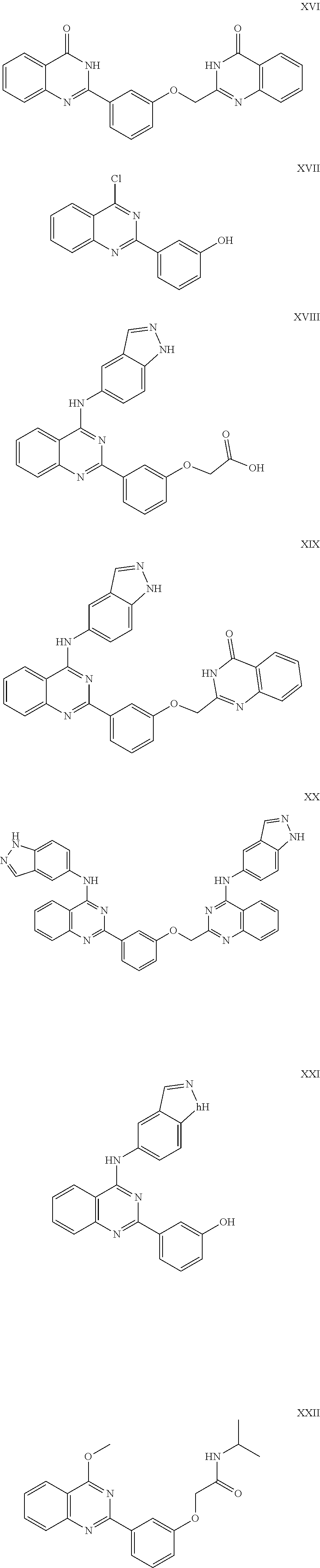

In an embodiment, the belumosudil mesylate, the compound I-A that is substantially free of impurities i.e. the compounds XI to XXII is crystalline belumosudil mesylate obtained by the processes of the present invention.

The impurities set forth above can be detected by standard analytical techniques known to a person of skill in the art such as High Performance Liquid Chromatography (HPLC), Mass Spectrometry (MS) and the like.

As used herein, the term “substantially free of impurities”, refers to the belumosudil mesylate obtained by the process of the present invention having less than from about 0.15% w/w to about 0.03% w/w (as determined by HPLC) of the above specified impurities.

In one embodiment, the isolated belumosudil mesylate has a purity ≥95% as measured by HPLC.

In an aspect, the present invention provides a pharmaceutical composition comprising crystalline belumosudil mesylate obtained by any one of the processes herein described, and a pharmaceutically acceptable excipient.

In an embodiment, the crystalline belumosudil mesylate contained in the pharmaceutical composition of the present invention may have D₉₀ particle size of less than about 250 microns, preferably less than about 150 microns, more preferably less than about 50 microns, still more preferably less than about 20 microns, still more preferably less than about 15 microns, and most preferably less than about 10 microns: wherein the crystalline belumosudil mesylate may be prepared by any one of the processes recited herein above.

The particle size disclosed herein can be obtained by, for example, any milling, grinding, micronizing or other particle size reduction methods known in the art to bring the solid state belumosudil mesylate into a desired particle size range as described above.

The examples that follow are provided to enable one skilled in the art to practice the invention and are merely illustrative of the invention. The examples should not be read as limiting the scope of the invention.

### Examples

### General Methods:

### 1. HPLC Method (Instrumental Settings):

High performance liquid chromatography (HPLC) was performed with the conditions described below for detecting chemical purity:

Column: YMC Triart C18, 150×4.6 mm, 5; Column Temperature: 50° C., Mobile phase: Mobile Phase A; 0.001% Perchloric in water: Mobile Phase B=Acetonitrile:Methanol (40:60 v/v); Sample Cooler temperature: 10° C.;

| Time (min) | % Mobile Phase A | % Mobile Phase B |
|---|---|---|
| 0.01 | 70 | 30 |
| 05 | 70 | 30 |
| 35 | 20 | 80 |
| 47 | 20 | 80 |
| 48 | 70 | 30 |
| 58 | 70 | 30 |

Diluent-: 1) Water:Acetonitrile:Dimethyl Sulfoxide (50:50:1, v/v)
Diluent-: 2) Water:Acetonitrile (50:50, v/v)
Flow Rate: 1.0 mL/Minute, Detection wavelength: UV 258 nm; Injection Volume: 10 μL; Run time: 58.0 minutes.

### 2. Gas Chromatography (GC) (Instrumental Settings):

Gas chromatography (GC) was performed on Perkin Elmer Clarus 590instrument equipped with FID and Turbomatrix 40HS.

Column: DB-1, 60 m×0.32 mm; Oven Temperature: 40° C.-150° C., Injector/detector temperature: 220° C./270° C.: Carrier gas: Nitrogen, Diluent: Benzyl alcohol.

### 3. X-Ray Powder Diffraction (XRPD) (Instrumental Settings):

The measurements were performed on Philips X-Ray Diffractometer model XPERT-PRO (PANalytical) Detector: X'celerator [1] using Cu lamp with type and wavelength of the X-ray radiation: K-α₁ 1.54060 [Å], K-α₂ 1.5444 [Å] under the following conditions: The measurements were carried out with a Pre FIX module programmable divergence slit and anti-scatter Slit (Offset) 0.00°: Generator settings: 40 mA/45 kV, tube current 40 mAmp Time per step: 50 s, Step size: 0.0167, Peak width 2.00 and start angle (°) 2.0 and End angle: 50.0; Scan type: continuous; measurement performed at 25° C. The XRPD instrument is calibrated using NIST SRM 6-40C silicon standard and NIST SRM 1976 Alumina.

Sample preparation: Take an adequate amount of the sample to fill the sample holder using back-loading technique. Then load the sample holder between the X-ray optics-path and scan using the above described parameters. Integrate the obtained powder X-ray diffraction profiles using X'Pert HighScore Plus Software.

### 4. Thermogravimetric Analysis (Instrumental Settings):

TGA thermogram was recorded using TGA-Q500 (Waters). About 5-10 mg of sample was taken in sample holder and loaded it in furnace. The sample was heated up to 250° C. at the ramp rate of 10° C./min and the thermogram was integrated by using Universal V4.5A software and calculate the weight loss by sample up to 100° C.

### 5. Differential Scanning Calorimetry (Instrumental Settings):

The DSC thermogram was measured by a Differential Scanning calorimeter (DSC 822, Mettler Toledo) at a scan rate of 10° C. per minute in the temperature range of range is “30° C. to 300° C.”. The DSC module was calibrated with Indium and zinc standard. Method: An empty aluminium standard 40 μl pan was taken and put on the microbalance. Tared and weighed approximately about 2.0 mg of sample. The cover or lid of the pan was slightly pierced and sealed. The sample pan was placed in the left position of mark ‘S’ and empty pan was placed in the right position on mark ‘R’ of the DSC sensor. The furnace lid was placed and method was selected. Run the sample.

### Examples

### Example 1: Preparation of 2-chloro-N-(propan-2-yl)acetamide (compound II)

Chloroacetyl Chloride was added to a cooled solution of isopropyl amine in dichloromethane (MDC). After stirring the reaction mixture at a temperature of about 20-30° C., water was added. After separating the layers, the MDC layer was washed with aqueous sodium chloride solution, and methyl isobutyl ketone (MIBK) was added to the organic layer. The organic layer was heated, and MDC was distilled, followed by partial distillation of MIBK under reduced pressure to obtain a solution of compound II in MIBK. Purity >95.0% (as determined by GC).

### Example 2: Preparation of methyl 3-[2-(isopropylamino)-2-oxoethoxy]benzoate (Compound IV-A)

A reaction mixture of 2-chloro-N-(propan-2-yl)acetamide (compound II) as a solution in MIBK, methyl 3-hydroxybenzoate (III-A), potassium carbonate and MIBK were refluxed for 12-14 hrs. The reaction mixture was cooled to temperature of about 20° C. to 30° C., and water was added to it. The layers were separated, and the organic layer was distilled under vacuum to get concentrated solution of compound IV-A in MIBK. Purity >95.0% (as determined by HPLC).

### Example 3: Preparation of 3-[2-(isopropylamino)-2-oxoethoxy]benzoic acid (Compound V)

To the cooled solution of methyl 3-[2-(isopropylamino)-2-oxoethoxy]benzoate (compound IV-A) in MIBK and methanol, aqueous sodium hydroxide was added, and stirred at temperature of about 20° C. to 30° C. Water was added to the reaction mixture. The layers were separated and to the aqueous layer, dilute hydrochloric acid solution was added to obtain a pH of about 1 to 3. After filtration, the product was dried to obtain compound V as a white solid. Purity >95.0% (as determined by HPLC).

### Example 4: N-[2-(aminocarbonyl)phenyl]-3-[2-(isopropylamino)-2-oxoethoxy]benzamide Compound (VII)

Method 1. To a cooled suspension of 3-[2-(isopropylamino)-2-oxoethoxy]benzoic acid (compound V) in dichloromethane, 2-aminobenzamide and catalytic amount of dimethylamino pyridine was charged followed by addition of EDC·HCl to obtain a reaction mass. The reaction mass was stirred at a temperature of about 20° C. to 30° C. To the reaction mass, water was added and stirred. The reaction mass was filtered. The resulting wet material was used in the next step. Purity 95% (as determined by HPLC).

Method 2: To the cooled suspension of 3-[2-(isopropylamino)-2-oxoethoxy]benzoic acid (compound V) in dichloromethane, catalytic amount of dimethyl formamide was added followed by addition of thionyl chloride. The reaction mass was stirred at ambient temperature. The excess thionyl chloride was distilled to obtain Compound V-A. To the residue, dichloromethane and 2-aminobenzamide were added to obtain a reaction mixture. The reaction mixture was cooled to a temperature of about 0° C. to about 10° C., and to the reaction mixture diisopropyl ethyl amine was added slowly. The resulting mixture was stirred at ambient temperature. Water was added to reaction mixture and then filtered. After drying at a temperature of about 55° C. to 60° C., the compound VII was obtained as off white solid. Purity >90.0% (as determined by HPLC).

### Example 5: Preparation of N-isopropyl-2-[3-(4-oxo-3,4-dihydroquinazolin-2-yl)phenoxy]acetamide (Compound VIII)

A mixture of wet N-[2-(aminocarbonyl)phenyl]-3-[2-(isopropylamino)-2-oxoethoxy]benzamide (compound VII), isopropyl alcohol (IPA), water and potassium carbonate were heated at a temperature range of about 75° C. to 85° C. To the cooled reaction mixture, water was added and filtered. After drying at a temperature of about 55° C. to 60° C., the compound VIII was obtained as an off-white to cream coloured solid. Purity >95% (as determined by HPLC).

### Example 6: Preparation of 2-[3-(4-chloroquinazolin-2-yl)phenoxy]-N-isopropylacetamide (Compound IX)

A mixture of N-isopropyl-2-[3-(4-oxo-3,4-dihydroquinazolin-2-yl)phenoxy]acetamide (compound VIII, 100.0 g) in dimethyl formamide (DMF, 1000 ml) was cooled to a temperature of about −8° C. to 0° C. To this a mixture of thionyl chloride (3.0 mole equivalent) and DMF (3.0 mole equivalent) was charged and stirred at same temperature. After completion of reaction, the reaction mixture was added to a cooled mixture of water and MDC. The layers were separated and MDC layer was washed with water and aqueous sodium bicarbonate solution. Aqueous sodium bicarbonate solution was washed with MDC. To the combined MDC layers, triethylamine was added. The organic layer was distilled under vacuum and IPA stripping was given. To the slurry mass IPA was added. The obtained solution was heated to a temperature of about 55 to 60° C. for about 15 min to 60 min, cooled to 20° C. to 30° C. and filtered. The product was dried at 40 to 50° C. to obtain the compound IX as an off-white to cream colour solid. Purity >99% (as determined by HPLC).

### Example 7: Preparation of 2-(3-{4-[(1H-indazol-5-yl)amino]quinazolin-2-yl}phenoxy)-N-(propan-2-yl)acetamide (compound I)

The reaction mixture of 2-[3-(4-chloroquinazolin-2-yl)phenoxy]-N-isopropylacetamide (compound IX, 100.0 g), 5-amino indazole (41.20 g), DIPEA (diisopropylethylamine, 40 g) in IPA (500 ml) was heated at a temperature of about 75° C. to 85° C. The reaction mixture was then cooled to a temperature of about 20° C.-30° C. followed by addition of water. The reaction mixture was then filtered. The wet material was slurried in MDC and filtered. After drying at temperature of about 55° C. to 60° C. the compound I was obtained as brown colour solid. Purity >99% (as determined by HPLC).

### Example 8: Preparation of 2-(3-{4-[(1H-indazol-5-yl)amino]quinazolin-2-yl}phenoxy)-N-(propan-2-yl)acetamide methane sulfonate Salt (I-A Wherein HA is Methane Sulfonic Acid)

The reaction mixture of 2-(3-{4-[(1H-indazol-5-yl)amino]quinazolin-2-yl}phenoxy)-N-(propan-2-yl)acetamide (compound I), methane sulphonic acid and methanol was stirred at a temperature of about 20° C. to 30° C. The reaction mixture was cooled and filtered. Purity 96.4% (as determined by HPLC).

To the above wet material, ethanol and water were charged and heated to a temperature of about 70° C. to 75° C. Then cooled to a temperature of about 20° C.-30° C., and filtered. The product was dried at a temperature range of about 55° C. to 60° C. to get compound I-A. Purity >99% (as determined by HPLC).

### Example 9: Preparation of 2-(3-{4-[(1H-indazol-5-yl)amino]quinazolin-2-yl}phenoxy)-N-(propan-2-yl)acetamide methane sulfonate Salt (I-A Wherein HA is Methane Sulfonic Acid)

The reaction mixture of 2-(3-{4-[(1H-indazol-5-yl)amino]quinazolin-2-yl}phenoxy)-N-(propan-2-yl)acetamide (compound I) and 1,4-Dioxane was heated to a temperature of about 70° C. to 75° C. Methane sulphonic acid was added to the reaction mixture and stirred at a temperature of about 70° C. to 75° C. The reaction mixture was cooled to temperature of about 20° C. to 25° C. stirred and filtered. After drying at a temperature range of about 55° C. to 60° C. compound I-A was obtained.

### Example 10: Preparation of 2-(3-{4-[(1H-indazol-5-yl)amino]quinazolin-2-yl}phenoxy)-N-(propan-2-yl)acetamide methane sulfonate Salt (I-A Wherein HA is Methane Sulfonic Acid)

To the reaction mixture of 2-(3-{4-[(1H-indazol-5-yl)amino]quinazolin-2-yl}phenoxy)-N-(propan-2-yl)acetamide (compound I), ethanol (15V), water (5V) and methane sulphonic acid were added, and stirred at temperature of about 20° C.-25° C. The reaction mixture was heated to a temperature of about 48° C. to 52° C. stirred and cooled. The reaction mixture was filtered and dried at a temperature of about 45° C. to 50° C. to obtain belumosudil mesylate. Purity 99.84% (as determined by HPLC).

### Example 11: Preparation of 2-(3-{4-[(1H-indazol-5-yl)amino]quinazolin-2-yl}phenoxy)-N-(propan-2-yl)acetamide methane sulfonate salt (I-A Wherein HA is Methane Sulfonic Acid)

Method 1: To the belumosudil mesylate obtained in example 10, o-xylene was added to obtain a reaction mixture, and the reaction mixture was heated to a temperature of about 144° C., and water was removed azeotropically. The reaction mixture was cooled to a temperature of about 50° C., and the resulting belumosudil mesylate obtained as solid was filtered. The belumosudil mesylate was dried at a temperature range of about 55° C. to 60° C. to obtain crystalline belumosudil mesylate. Purity 99.78% (as determined by HPLC).

Method 2: Methane sulphonic acid (1.0 mole equivalent) was added to the reaction mixture of 2-(3-{4-[(1H-indazol-5-yl)amino]quinazolin-2-yl}phenoxy)-N-(propan-2-yl)acetamide (compound I), o-xylene (5V) and water (5V). This was followed by stirring the reaction mixture at a temperature of about 20° C. to 25° C. The reaction mixture was heated to a temperature of about 90° C. to 100° C. and stirred for 50 mins. The reaction mixture was further heated to a temperature of about 144° C., stirred for about 1 hr and water was removed azeotropically during the heating operation. The reaction mixture was cooled to a temperature of about 50° C. to 55° C., filtered and dried at a temperature of about 55° C. to 60° C. under vacuum to obtain crystalline belumosudil mesylate. Purity>99.64% (as determined by HPLC).

### Example 12: Preparation of 2-(3-{4-[(1H-indazol-5-yl)amino]quinazolin-2-yl}phenoxy)-N-(propan-2-yl)acetamide methane sulfonate Salt (Compound I-A)

To the reaction mixture of 2-(3-{4-[(1H-indazol-5-yl)amino]quinazolin-2-yl}phenoxy)-N-(propan-2-yl)acetamide (compound I, 100.0 g) and dimethyl sulfoxide (DMSO, 5 V) was added methane sulphonic acid (1.0 mole equivalent) and stirred at a temperature of about −5° C. to 5° C. The reaction mixture was filtered through micron filter. To the clear filtrate methyl isobutyl ketone (MIBK, 10 V) was added and stirred at a temperature of about 20° C. to 25° C. for a period of about 3 hrs. The reaction mass was filtered and washed with water. Dimer impurity (XXI): 0.11%, Methoxy impurity (XXII): 0.14% and other impurity (XIX): 0.02%.

To the above wet material ethanol (7.5V) and water (2.5V) was added to obtain a reaction mass. The reaction mass was heated to a temperature of about 70° C. to 75° C. and stirred for 30 mins. The reaction mixture was cooled to a temperature of about 20° C. to 30° C. and stirred for about an hour. The reaction mass was filtered, and the resulting product dried at a temperature range of about 55° C. to 60° C. under vacuum to obtain the compound I-A.
Purity >99.89% (as determined by HPLC).
Impurity XVI, Impurity XVIII and Impurity XIX: Not detected;
Dimer impurity XX: 0.02 to 0.04%;
Impurity XXI: 0.01 to 0.02%;
Methoxy impurity XXII: 0.06%.

1. A process for the preparation of belumosudil, a compound of formula I, or a pharmaceutically acceptable salt thereof, 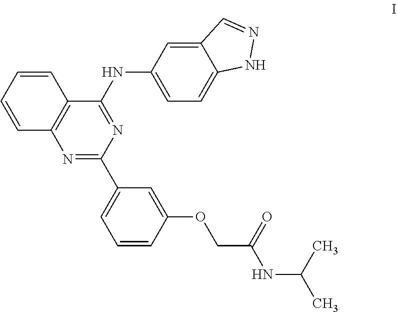 [SMILES: CC(C)NC(=O)COc1cccc(-c2nc(Nc3ccc4[nH]ncc4c3)c3ccccc3n2)c1]
comprising the steps of:
a) reacting chloroacetyl chloride with isopropyl amine to obtain 2-chloro-N-(propan-2-yl)acetamide, a compound of formula II (the “compound II”); 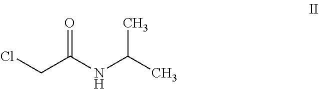
b) reacting the compound II with an alkyl ester of 3-hydroxy benzoate (the “compound III”) in the presence of a base to obtain alkyl ester of 3-[2-(isopropylamino)-2-oxoethoxy]benzoate, a compound of formula IV (the “compound IV”); 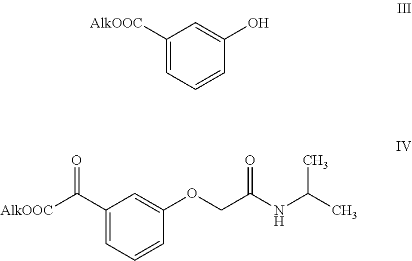
wherein Alk refers to alkyl;
c) reacting the compound IV with an acid or a base to obtain 3-[2-(isopropylamino)-2-oxoethoxy]benzoic acid, the compound of formula V (the “compound V”); 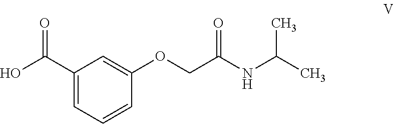 [SMILES: CC(C)NC(=O)COc1cccc(C(=O)O)c1]
d) reacting the compound V with 2-aminobenzamide (the “compound VI”) in the presence of a coupling agent to obtain N-[2-(aminocarbonyl)phenyl]-3-[2-(isopropylamino)-2-oxoethoxy]benzamide, a compound of formula VII (the “compound VII”); 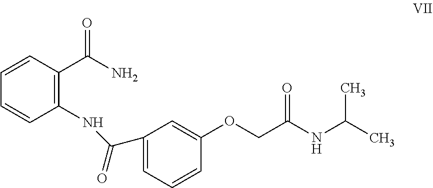 [SMILES: CC(C)NC(=O)COc1cccc(C(=O)Nc2ccccc2C(N)=O)c1]
e) reacting the compound VII with a base to obtain N-isopropyl-2-[3-(4-oxo-3,4-dihydroquinazolin-2-yl)phenoxy]acetamide, a compound of formula VIII (the “compound VIII”); 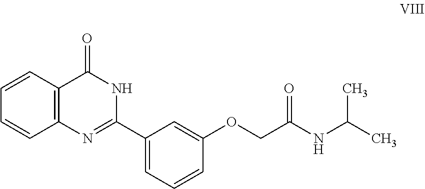 [SMILES: CC(C)NC(=O)COc1cccc(-c2nc3ccccc3c(=O)[nH]2)c1]
f) reacting the compound VIII with a chlorinating agent to obtain 2-[3-(4-chloroquinazolin-2-yl)phenoxy]-N-isopropylacetamide, a compound of formula IX (the “compound IX”); 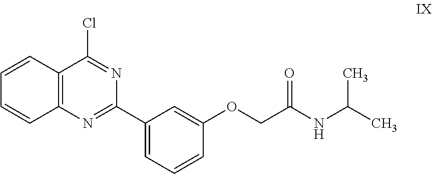 [SMILES: CC(C)NC(=O)COc1cccc(-c2nc(Cl)c3ccccc3n2)c1]
g) reacting the compound IX with 5-amino indazole to obtain belumosudil, the compound I; and
h) optionally, converting belumosudil to its pharmaceutically acceptable salt.

2. The process according to claim 1, wherein the coupling agent used in the step d) is selected from the group consisting of N-Ethyl-N′-(3-dimethylaminopropyl) carbodiimide hydrochloride (EDC·HCl), N,N′-Dicyclohexylcarbodiimide (DCC), 1-Hydroxybenzotriazole (HOBT), 1-[Bis(dimethylamino)methylene]-1H-benzotriazolium 3-oxide tetrafluoroborate (TBTU), (2-(1H-benzotriazol-1-yl)-1,1,3,3-tetramethyluronium hexafluorophosphate (HBTU), and (1-[Bis(dimethylamino)methylene]-1H-1,2,3-triazolo[4,5-b]pyridinium 3-oxide hexafluorophosphate (HATU).

3. The process according to claim 1, wherein the chlorinating agent is selected from the group consisting of thionyl chloride, phosphorous oxychloride and phosphorous pentachloride.

4. The process according to claim 1, wherein in the step h) belumosudil is converted to its methane sulfonate salt.

5. A one-pot process for the preparation of pharmaceutically acceptable salt of belumosudil, a compound of formula I-A (the “compound I-A”); 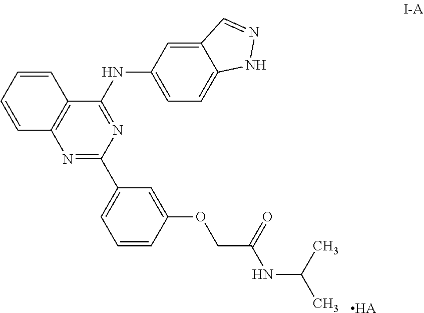
wherein HA is an acid selected from the group consisting of trifluoroacetic acid, methane sulfonic acid, hydrochloric acid, hydrobromic acid, oxalic acid, benzenesulfonic acid, p-toluenesulfonic acid, citric acid, tartaric acid, sulphuric acid, maleic acid, and fumaric acid;
comprising the steps of:
i) reacting the compound of formula IX (the “compound IX”) with 5-aminoindazole 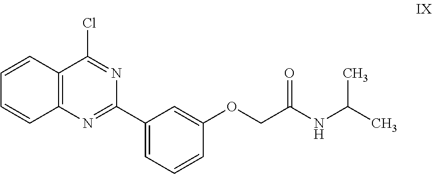 [SMILES: CC(C)NC(=O)COc1cccc(-c2nc(Cl)c3ccccc3n2)c1]
to obtain a reaction mixture containing belumosudil free base, the compound I; and
(ii) reacting the reaction mixture containing the belumosudil free base obtained in step (i) with an acid HA to obtain the pharmaceutically acceptable salt of belumosudil, the compound I-A;
wherein belumosudil, the compound I, obtained in step i) is carried forward without isolation for reaction with an acid in step ii).

6. The process according to claim 5, wherein the acid HA used in step (ii) is methane sulfonic acid to obtain belumosudil methane sulfonate salt.

7. A process for the preparation of crystalline belumosudil mesylate, the compound I-A, said process comprising the steps of:
(1-a) treating belumosudil mesylate with a mixture of water and a solvent to obtain a reaction mixture;
(1-b) heating the reaction mixture obtained in step (1-a);
(1-c) cooling the reaction mixture obtained in step (1-b); and
(1-d) isolating crystalline belumosudil mesylate from the reaction mixture of step (1-c); wherein the crystalline belumosudil mesylate is characterised by X-ray powder diffraction (XRPD) spectrum having peak reflections at about 7.1, 17.2, 20.3, 21.5, 22.1, 25.0 and 25.5±0.2° θ.

8. The process as claimed in claim 7, wherein the solvent is (C₁-C₆) alcohol selected from the group consisting of methanol, ethanol, n-propanol, t-butanol, n-butanol, and isopropanol.

9. (canceled)

10. (canceled)

11. (canceled)

12. (canceled)

13. (canceled)

14. (canceled)

15. The process according to claim 7, wherein belumosudil methane sulfonate is substantially free of impurities represented by the following compounds of formulae XI to XXII; [chemical structure]
